(19) United States
(12) Patent Application Publication
(10) Pub. No.: US 2006/0261180 A1
(43) Pub. Date: Nov. 23, 2006

(54) Device for impregnating arrow fletchings with scent

(76) Inventor: Steve Tabb, Springfield, IL (US)

Correspondence Address: National IP Rights Center, LLC;Suite 400, 550 Township Line Road, Blue Bell, PA, 19422, US

(21) Appl. No.: 11/091,085

(22) Filed: Mar. 28, 2005

(51) Int. Cl. A24F 25/00; A61L 9/04

(52) U.S. Cl. 239/34; 239/54; 239/55; 239/60

(57) ABSTRACT

A device and method of applying game scents to the fletchings of an arrow is disclosed. The scent applying devices included an absorbent material or spraying device, which assist in the transfer of specific scent liquids to an arrow fletching.

### FIELD OF THE INVENTION

The present invention is directed to device for impregnated arrow fletchings with a scent, for use in attracting game for bow hunting.

### BACKGROUND OF THE INVENTION

In the sport of bow and arrow game hunting, scents are often used to lure game into a desired location. If a hunter exits his hiding spot to distribute this attractant, there is a substantial chance that local game will be startled by the human scent and noise created by this movement. Therefore, a scent impregnated projectile, which can be strategically shot into a desired location, will improve the chances of attracting game. As a result, numerous scent emitting arrows have been introduced into the market to aid hunters.

U.S. Pat. No. 5,035,435 discloses a hunting scent dispersing device for attachment to the shaft of an arrow. The dispersing device includes an absorbant carrier for retaining and permitting release therefrom of a hunting scent which is to be releasably attached to the arrow.

U.S. Pat. No. 6,450,905 discloses a scent dispersion apparatus for delivery via an arrow shot by a bow. The apparatus uses an arrow tip using a compression fitting for attaching to the arrow shaft. A scent dispersion head slidably fits over the arrow tip, and contains a sponge which holds liquid scent. The arrow tip acts like a plunger to force the scent liquid out upon target impact. The arrow tip includes O-rings to hold the dispersion head in place until target impact.

U.S. Pat. No. 6,174,251 discloses an arrow intended for dispersing olfactant (for example, for use in deer hunting) and comprising an elongate body having an internal cavity, this cavity having a container portion to accommodate a frangible liquid container. A container rupturing device is disposed within the cavity adjacent the container portion, this device being such that the arrow, with a container on board, can be launched from a bow without rupturing the container, but that upon the arrow striking a target, the device will rupture the container. A wick is disposed within the cavity adjacent the container portion, this wick being capable of absorbing the liquid released from the rupture of the container. The body has apertures extending from the cavity to the exterior of the body adjacent the wick, so that gas produced by evaporation of liquid absorbed on the wick can diffuse out of the arrow.

U.S. Pat. No. 5,836,842 discloses an arrow for dispersing scent to attract game to be used by a bow hunter from a remote location. Said scent dispersing arrow having an improved means for holding the desired animal scent and for dispensing said scent upon the arrow's impact with a solid surface. Said arrow having a cylindrical cartridge for retaining the scent mounted within the forward end of said arrow's tubular shaft. The forward arrow end also having a two-piece containment means with a blunt pointed end with a rearward extending cylindrical piston, said piston pushing into the cylindrical cartridge upon impact and expelling the liquid scent through a passageway into the area of the arrow's impact, the arrow having an attachment means at its rearward end for a thin line, unreeled in flight, by which the arrow may be retrieved by the bow hunter, without leaving his position.

One of the problems associated with these types of systems is that they specifically require replacement or modification of a hunter current arrow collection. It would be desirable to provide a system whereby the fletchings of an arrow could be easily impregnated with a specific scent. Such a system would a hunter to quickly customize any arrow within his quiver with the appropriate scent for the specific game being hunted.

### OBJECTS AND SUMMARY OF THE INVENTION

It is an object of the present invention to provide a scent applying device that allows bow and arrow game hunters to apply a scent to their current collection of arrows.

It is a further object of the present invention to provide a scent applying device that can be easily carried and used by hunters.

It is a further object of the present invention to provide specific scent applying devices to assist in attracting various game.

It is a further object of the present invention to provide a system in which scents can be applied directly to an arrow fletchings.

It is a further object of the present invention to provide hunters with the ability to equip themselves with scent attracting arrows, without having to replace their current collection.

In accordance with a first aspect of the present invention, a device for impregnating arrow fletchings with a specific game attracting scent is disclosed. The scent applicator is a handheld device, wherein an arrow fletching is rubbed against the device and scent is applied to said fletching.

In a further embodiment, the invention is an arrow fletching scent applying device, comprising: an absorbent material having a scent applied thereto for placement on the fletchings of an arrow. The absorbent material may comprise a fabric, sponge, or foam material.

In still a further embodiment, the invention is an arrow fletching scent applying device, comprising: a block shaped liquid scent reservoir having a groove and a plurality of apertures within the groove, said groove being configured to receive a fletching, said fletching receiving scent from the apertures.

In yet a further embodiment, the invention is a method for applying specific scents to the fletchings of an arrow, comprising the step of transferring the liquid from the scent application device to the fletchings of an arrow.

### BRIEF DESCRIPTION OF THE FIGURES

The foregoing summary, as well as the following detailed description of a preferred embodiment of the present invention will be better understood when read with reference to the appended drawings, wherein:

FIG. 1 is a perspective view of a cloth scent applicator in accordance with the present invention.

FIG. 2 is a perspective view of a spraying device for scent application in accordance with the present invention.

FIG. 3 is a perspective view of a handheld device for scent application in accordance with the present invention.

FIG. 4 is a partial cross section of the handheld device of FIG. 3.

### DETAILED DESCRIPTION OF THE PREFERRED EMBODIMENT

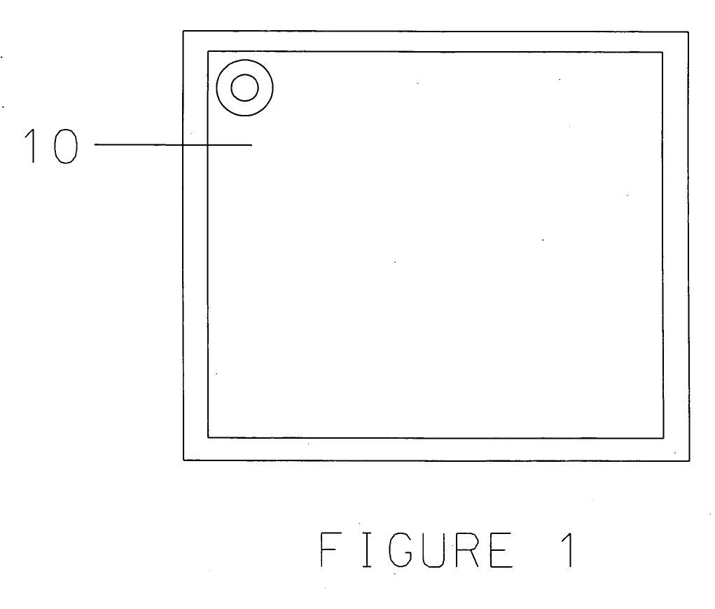

The present invention is directed to a system and method for impregnating the fletchings of an arrow with a specific game attracting scent. Referring now to the drawings wherein like reference numerals refer to the same components across the several views, and in particular to FIG. 1, there is shown a cloth scent applicator 10. The cloth scent applicator comprises an absorbent fabric which may be constructed from cotton or synthetic fabric.

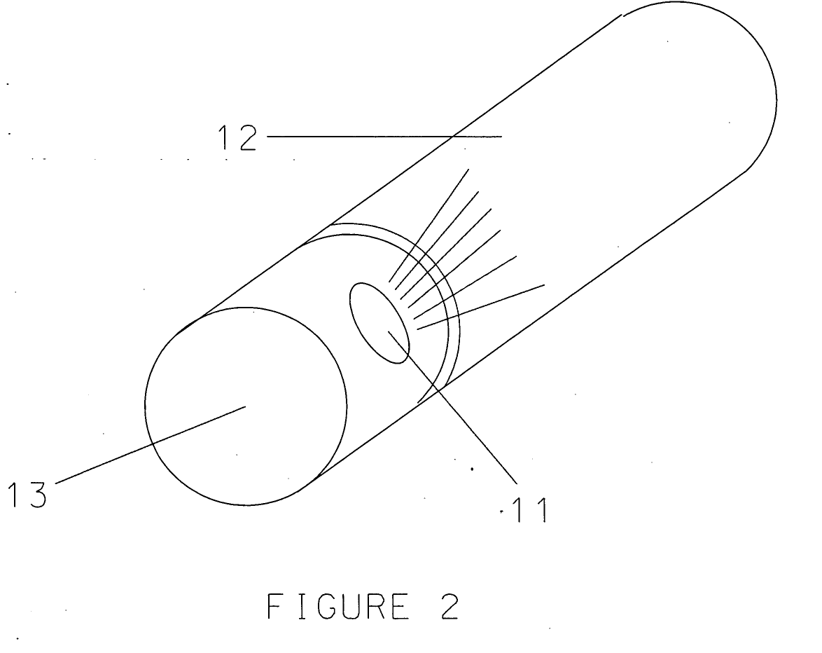

Continuing to FIG. 2, a preferred method of using the cloth scent applicator 10 will be described. The cloth square 10 would be dampened with a particular scent. The cloth would then be rubbed along the fletchings on an arrow.

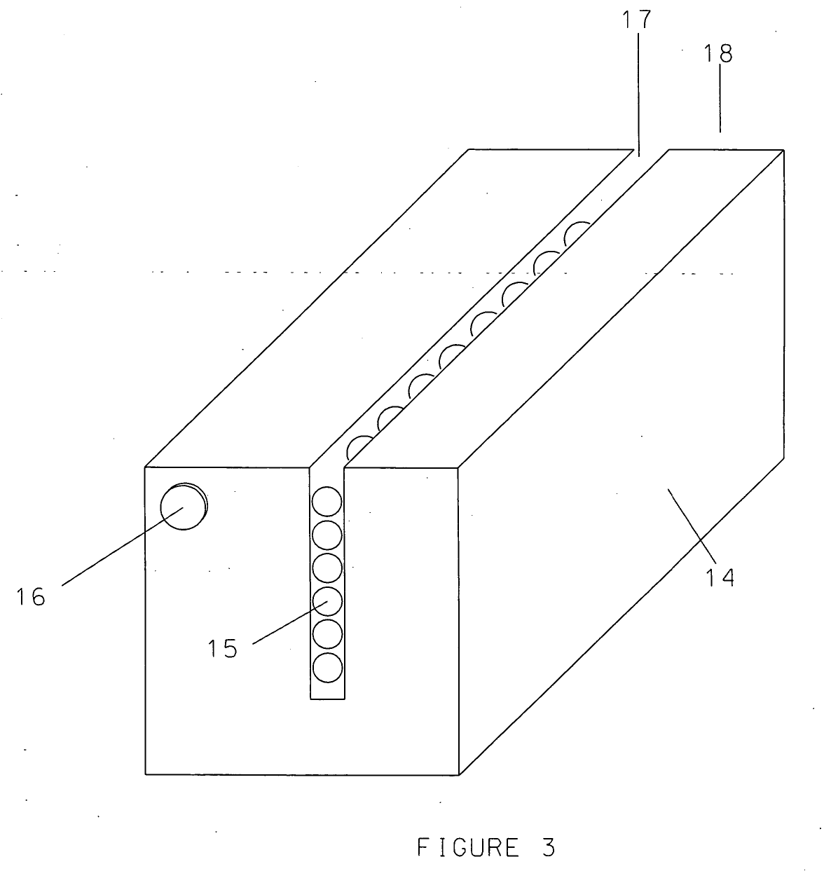

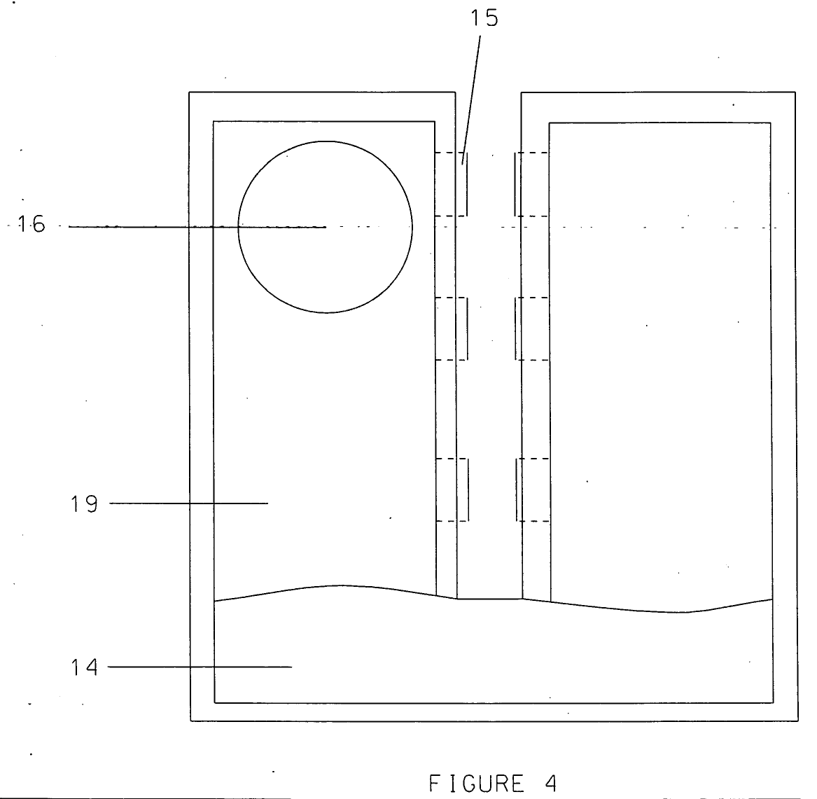

FIGS. 3 and 4 illustrate still a further embodiment. In this embodiment, the system comprise an applicator system. The system comprises a block 14 having a central groove 17 with applicator tips 20. A scent is poured into an aperture 16. The block 14 is hollow to store liquid scent 19 and the tips 20 have small apertures 20a to pass scent onto the fletchlings. When the fletching are passed within the groove, they are coated with the scent.

In view of the foregoing disclosure, some of the advantages of the present invention can be seen. For example, a cloth scent applicator and method are disclosed. The cloth scent applicator can easily apply a game scent to a set of arrows owned by a hunter. Hunters, therefore, do not need to purchase new scent equipped arrows to attract game to a specific area.

In still a further embodiment, the invention envisions an embodiment in which the fletchings are permanently pre-impregnated with scent at the time of manufacture. This embodiment eliminates the need to add scent to the fletchings in the field.

While the preferred embodiments of the present invention have been described and illustrated, modifications may be made by one of ordinary skill in the art without departing from the scope and spirit of the invention as defined in the appended claims. For example, with regard to the scent apply device, a cloth applicator is disclosed, however, more complex fletching spraying or rubbing devices may be employed. Likewise, although the preferred method described rubbing scents onto the fletchings of an arrow, the hunter may also spray or dip said fletchings.

1. An arrow fletching scent applying device, comprising:
(a) an absorbent material having a scent applied thereto for placement on the fletchings of an arrow.

2. The arrow fletching scent applying device of claim 1, where said material comprises a fabric, sponge, or foam material.

3. An arrow fletching scent applying device, comprising;
(a) a handheld container;
(b) an absorbent material within said container.

4. The arrow fletching scent applying device of claim 3, where said material comprises a fabric, sponge, or foam material

5. The handheld container of claim 3, where said container has inserts so an arrows fletching may contact said material.

6. The handheld container of claim 3, where said container has a reservoir, when scent liquid may be stored and transferred to said material.

7. An arrow fletching scent applying device, comprising:
a block shaped liquid scent reservoir having a groove and a plurality of apertures within the groove, said groove being configured to receive a fletching, said fletching receiving scent from the apertures.

8. A method of applying specific scents to the fletchings of an arrow, comprising the step of:
(a) transferring the liquid from the scent application device to the fletchings of an arrow.
